(19) United States
(12) Patent Application Publication
(10) Pub. No.: US 2007/0173952 A1
(43) Pub. Date: Jul. 26, 2007

(54) Implant and method of producing the same, and a system for implantation

(75) Inventors: Leif Hermansson, Molle (SE); Hakan Engqvist, Knivsta (SE); Jesper Loof, Uppsala (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(73) Assignee: DOXA AB, Uppsala (SE)

(21) Appl. No.: 10/580,613

(22) PCT Filed: Nov. 25, 2004
(86) PCT No.: PCT/SE04/01745

§ 371 Date: Mar. 8, 2007

(30) Foreign Application Priority Data
Nov. 27, 2003 (SE) 0303169-7

(51) Int. Cl. A61F 2/02

(52) U.S. Cl. 623/23.76; 523/115; 623/23.6; 427/2.27

(57) ABSTRACT

The present invention relates to a coated implant for in vivo-anchoring of implants to a biological tissue or another implant, which coated implant comprises an implant having a pre-treated surface and on said pre-treated surface one or more layers of ceramic material chemically and/or mechanically bound to said pre-treated surface. Said one or more layers comprises mainly non-hydrated chemically bonded ceramic material, and each layer independently comprises a first binder phase selected from the group consisting of aluminates, silicates, phosphates, sulphates and combinations thereof. The invention further relates to method of manufacturing said coated implant, a ceramic paste and to a kit comprising said coated implant and ceramic paste. The invention is particularly suitable for dental and orthopaedic implants.

### TECHNICAL FIELD

The present invention relates to a coated implant for in vivo-anchoring of implants to a biological tissue or another implant, which coated implant comprises an implant having a pre-treated surface and on said pre-treated surface one or more layers of ceramic material chemically and/or mechanically bound to said pre-treated surface. The invention further relates to method of manufacturing said coated implant, and to a kit comprising said coated implant and a ceramic paste comprising a calcium-based binder. The invention is particularly suitable for dental and orthopaedic implants.

### STATE OF THE ART AND PROBLEM

For implants that are to interact with the human implant, it is an advantage with implant materials that due to their biocompatibility provide an optimal fixation or anchoring of the implant to the biological tissue, e.g. bone. Even small gaps may lead to small movements, micromotions, between implant and the tissue, which increase the risk of implant loosening, e.g. due to formation of zones of fibrous tissue at the implant-tissue interface. Porosity or cavities in the tissue surface (vacuoles) also reduce the implant fixation. To allow for early loading of an implant and to reduce the risk for long term loosening, high quality early fixation is important.

Also, in the case of a coated implant, the anchoring of the coating to the implant surface may be the weak point of the implant system.

The chemical systems used in the present invention are based on aluminate, silicate and/or phosphate systems of chemically bonded ceramics, CBC, the systems of which are intended for biomaterial applications earlier described in SE 463,493, SE 502,987, WO 00/21489, WO 01/76534, WO 01/76535, PCT/SE02/01480 and PCT/SE02/01481. An organic (polymeric) constituent may be added to the CBC materials and particularly to the material in the form of a paste as described in the co-pending patent application SE-A0-0302844-6. CBC materials used as coatings in pre-hydrated stage are also described in SE 521973, SE 522749 and SE 0203223-3.

### SUMMARY OF THE INVENTION

In view of the prior art implants for use in contact with biological tissue, particularly when anchoring implants in bone, there is a need for an implant and implant anchoring technique which provides a sufficiently high strength, and thus load-bearing capacity, shortly after application, as well later on, and which furthermore promotes re-growth of the bone.

To fulfil said needs, the present invention provides an implantation system comprising chemically bonded ceramics as main phase(s), which when cured in vivo, provides a sufficiently high strength. Said strength is achieved shortly after insertion of an implant coated with a ceramic material and optionally also a ceramic paste.

According to a first aspect, there is provided a coated implant for in vivo-anchoring to a biological tissue or another implant. The coated implant is defined in claim 1.

According to second aspect, there is provided a method of manufacturing said coated implant. Said method is defined in claim 21.

According to fourth aspect, there is provided a ceramic paste for enhancing the in vivo-anchoring of the implant. Said paste is defined in claim 31.

According to third aspect, there is provided an implantation kit for in vivo-anchoring an implant to a biological tissue or another implant, comprising said coated implant and said ceramic paste. The implantation kit is defined in claim 35.

The main advantages of the present invention is high early strength of the coating formed in vivo, which strength emanates from the strong adhesion of the coating to the implant surface and the anchoring of the coated implant in the designated tissue. The strength of the coating is a result of the selected chemically bonded ceramic material and the size of its particles and the pre-treatment of the implant surface. The rapid anchoring of the coated implant in the tissue is due to the fact that the coating comprises non-hydrated binder phases, for example calcium aluminate, which upon hydration takes up water, whereby the volume (or mass) of the points where the coating meets the tissue increases. This enlarges the implant's contact area with the surrounding tissue at an early stage, whereby the implant can be loaded early, and before the long-term anchoring occurs, as a result of new bone in-growth towards the implant.

The coated implant, ceramic paste and the implantation kit according to the invention are particularly suitable for orthopaedic and dental applications.

### DESCRIPTION OF DRAWINGS

In the following, the mechanism at implanting will be described in greater detail with reference to a preferred embodiment.

FIG. 1 shows a cross-sectional view of the outer part of a coated implant according to the present invention,

FIG. 2 shows a cross-sectional view of the part according to FIG. 1, provided with an extra, outermost layer, and a ceramic paste according to the present invention,

FIG. 3 shows a cross-sectional view of the coated implant, including the ceramic paste, according to FIG. 2 immediately after it has been arranged (implanted) against a biological wall,

FIG. 4 shows a cross-sectional view of the implant and paste according to FIG. 3, after about one hour,

FIG. 5 shows a cross-sectional view of the implant and paste according to FIG. 3-4 after healing,

FIG. 6 shows a high-resolution TEM picture (magnification 600.000 X) of the contact zone between a coating and a Ti-implant surface according to the present invention.

FIG. 7 shows a cross-sectional view of an image of hydrates formed after 24 h in rabbit femur, when using a coated implant according to the present invention.

### DETAILED DESCRIPTION OF THE INVENTION

The present invention aims at providing an implant coated with layers of chemically bonded ceramic materials (CBC-materials), for in vivo anchoring of an implant to a biological tissue, such as bone. The implant may be ceramic, polymeric or metallic. The system is characterized by:
a) Anchoring by hydration of a CBC-material to the surface of the pretreated implant and enhanced by chemical and/or mechanical treatment,
b) inter-anchoring of individual sub-layers of the CBC-material (by liquid transport and co-hydration),
c) anchoring of the CBC-material to a CBC-paste (by surface treatment and co-hydration),
d) anchoring of the CBC-paste (and the layered CBC-material) to the biological tissue (by dissolution-precipitation and volume increase).

Also, the coated implant, ceramic paste and implantation kit should fulfill requirements on implantation systems and materials, such as desired porosity and desired thickness to optimize the mechanical property profile, i.e. high shear strength of the inner layer towards the implant and reduced thickness of each individual layer to eliminate larger defects in the layers.

Such a coated implant is provided according to the invention as claimed. Said coated implant is suitable for in vivo-anchoring of an implant to a biological tissue or another implant. The coated implant comprises an implant having a pre-treated surface on said pre-treated surface one or more layers of a material with a phase having the capacity following wetting with a liquid to form a chemically bonded ceramic material. The material of said one or more layers is in the main non-hydrated prior to said in vivo-anchoring and said one or more layers have the capability to chemically and/or mechanically bind to said implant and optionally to a paste of a powdered material with a calcium-based binder phase having the capacity following wetting with a liquid reacting with it to form a chemically bonded ceramic material.

According to the invention, one or more of the layers and preferably at least the outermost layer is in the main non-hydrated. Following insertion of the coated implant into a living body, this/these layer(s) will hydrate by reaction with body liquid and/or any especially applied hydration liquid, for example provided by a paste of CBC-material applied onto the outermost layer and/or onto the biological tissue.

According to one embodiment of the invention, the implant surface is treated to a specific surface roughness. The surface treatment can be accomplished by e.g. a mechanical treatment such as sand blasting or grinding. The surface treatment may also be a chemical process such as etching including salt melts, oxidation including low-temperature oxidation with species such as ozone, Ca-enriched by surface diffusion and hydration. Through heat treatment of the implant in the presence of Ca, a chemically active surface layer can be formed, facilitating a better bond. The heat treatment is preferably performed at temperatures above 1000° C., even more preferably above 1300° C.

According to another embodiment of the invention, the surface roughness of the pre-treated surface of the implant has a Ra-value of less than 10, preferably less than 5 and more preferably less than 1 μm, but due to practical reasons not smaller than Ra=0.5 μm. Such a surface roughness has been found to be especially well adapted for the anchoring of an innermost CBC-material layer that is applied by a technique in the group that consists of thermal spraying, flame spraying, Electro Deposition Spraying (EDS), plasma spraying, dipping and spin coating.

According to another embodiment of the invention, the surface roughness of the pre-treated surface of the implant has a Ra-value of less than 1, preferably less than 0.5 and more preferably less than 0.1 μm, but due to practical reasons not smaller than Ra=0.05 μm. Such a surface roughness has been found to be especially well adapted for the anchoring of an innermost CBC-material layer that is applied by a technique in the group that consists of Chemical Vapor Deposition (CVD), Physical Vapor Deposition (PVD), laser techniques including laser cladding, Electrolytic Deposition (ED), and sol-gel technique. CVD, PVD or a sol-gel technique is especially preferred. The innermost layer of CBC-material should be relatively thin, i.e. thinner than any one of the other layers, in order to minimize mechanical stresses in that innermost layer. It is preferred that it has a thickness from the nanometer level to less than 10 μm, preferably smaller than 2.0 μm.

After a deposition of the one or more layers, some kind of thinning process of the layer may be beneficial, especially concerning but not limited to the innermost layer. The thinning process includes processes such as grinding and sand blasting or dry etching, but preferably chemical treatment including dissolution. In connection with the thinning a partial densification of the layer may be performed by techniques such as drying up of particles and precipitation including sol-gel techniques.

A mechanical anchoring of the first layer to the implant is achieved by the precipitation of sub-micron (nanometer) size crystallites of hydrates against the implant surface. The crystallite size is preferably below 100 nm, and more preferably below 50 nm. When using the method of manufacturing an implant according to the present invention, the size of the crystallites is generally 20-70 nm. The large surface area and thereby extremely high surface energy of such crystallites helps in anchoring the layer to the implant.

The innermost layer of CBC-material can also preferably be chemically bonded to the implant surface by a pre-treatment of said surface yielding a chemical change of the surface from the original metallic or ceramic character to an oxide, preferably a double oxide of titanate, silicate or aluminate type, of the original implant by treatment involving oxidation, calcination, ion bombarding or thermal pretreatment. In connection with the pre-treatment an inner layer of the CBC-material thus may be formed.

According to one embodiment of the invention, the number of layers of CBC-material are 1-8, preferably 1-5 and even more preferably 2-5. Each layer outside the innermost one independently has a thickness of less than 50 μm, preferably less than 30 μm, but not smaller than 5 μm. Before hydration, the layers should be relatively dense in terms of porosity, preferably having a porosity below 50% and even more preferably less than 20%. During the hydration, the porosity of the layers is reduced to less than 10%, preferably less than 5%. In the case of non-thermal deposition techniques, such as spin coating, dipping etc, however, a higher porosity than 50% is normally achieved.

Furthermore, it is preferred that each layer, including the innermost layer, independently has a binder phase in the group that consists of aluminates, silicates, phosphates, sulphates and combinations thereof, preferably having cations in the group that consists of Ca, Sr and Ba, calcium-based binder phases being preferred and calcium aluminates being most preferred, preferably having a composition comprising one or more of the phases 3CaO.Al₂O₃, 12CaO.7Al₂O₃, CaO.Al₂O₃, CaO.2Al₂O₃ and CaO.6Al₂O₃12CaO.7Al₂O₃ being the most preferred phase. The material can be in crystalline or amorphous state. Preferably, the powdered material has a particle size of 0.1 to 20 μm and more preferably 1 to 10 μm and most preferably 1 to 5 μm.

Accordingly, the different layers of the coating may be composed of different, or the same, CBC-material, hydrated to the same or to different degrees, although preferably no layer is completely hydrated before the implantation takes place. Hydration will take place, following implantation, by reaction with body liquid and/or any especially applied hydration liquid, for example provided by a paste of CBC-material applied onto the outermost layer and/or onto the biological tissue. Optionally and possibly in combination with the paste, an additional hydration liquid may be provided to the coating layers of the implant, before application of the paste and before implantation takes place, e.g. by dipping, spraying, spin coating or tape casting the coated implant in/with such an additional hydration liquid.

According to another aspect of the invention, the system also comprises a ceramic paste of a powdered material with a calcium-based binder phase of aluminate and/or silicate, having the capacity following wetting with a liquid reacting with the binder phase to hydrate to a chemically bonded ceramic material of any one of the above mentioned types, which powdered material is slurried in said liquid reacting with the binder phase to form said paste, said paste being capable of providing an in vivo-formed interface between said outermost layer and said biological tissue, and preferably having an initial viscosity, directly upon mixing and application of said powdered material and said liquid, of less than 100,000 cP, preferably less than 10,000 cP.

In one embodiment, an organic (polymeric) additive, preferably a hydrophilic polyacrylic and/or polycarboxylate compound, is added to the chemically bonded ceramic material and particularly to the paste. This organic additive is used to achieve suitable rheological properties, low water/cement-ratio and to act as a complementary binding system. This organic additive also imparts a more visco-elastic behavior to the ceramic materials, in addition to increased strength, as described in the co-pending patent application SE-A0-0302844-6.

Most beneficially, the powdered material of the paste has the form of granules, preferably of a size below 1 mm, more preferably below 0.5 mm and most preferably below 0.4 mm and having a granule compaction density above 35%, preferably above 50% more preferably above 60%.

By using granules the w/c ratio (water/cement ratio) can be lower than for the loose powder. The flow ability of the material is higher when it is granulated. By using highly compacted small granules, the shaping of the paste can take place in a subsequent step, without any remaining workability limitations of highly compacted bodies. A facilitated shaping in such a subsequent step, such as kneading, ultrasound etc., can be made while retaining a mobility in the paste system that has a high final degree of compaction, exceeding 35%, preferably exceeding 50%, even more preferably exceeding 60%.

According to one embodiment, the granules of the paste preferably exhibit a degree of compaction above 60%, even more preferably above 65% and most preferably above 70%. Preferably, the granules have a mean size of at least 30 μm, preferably at least 50 μm and even more preferably at least 70 μm, but 250 μm at the most, preferably 200 μm at the most and even more preferably 150 μm at the most, while the powder particles in the granules have a maximal particle size of less than 20 μm, preferably less than 10 μm. It should hereby be noted that it is only a very slight proportion of the powder particles that constitute particles having the maximal particle size. The particle size is measured by laser diffraction. The highly compacted granules are manufactured by the powdered material being compacted to the specified degree of compaction, by cold isostatic pressing, tablet pressing of thin layers, hydro-pulse technique or explosion compacting e.g., where after the material compacted accordingly is granulated, for example crushed or torn to granules of the specified size.

In the present anchoring system, the ceramic paste has the beneficial function of filling the gap between the implant and the biological tissue, and filling any vacuoles or cavities in the surface of the bone tissue. Also, due to its biocompability or bioactivity, it provides for an improved anchoring to the bone tissue and to the outermost layer of the coating, which outermost layer is surface treated in order to improve the anchoring to the paste and binding to the cured paste. Suitably, the surface of the outermost layer has a Ra-value less than 20 μm and even more preferably Ra less than 10 μm. However, especially in connection with an embodiment with only one single layer, most preferably applied by PVD technique, this layer preferably has a surface roughness with Ra<1 μm, more preferably Ra<0.5 μm and most preferably Ra<0.1 μm, but not smaller than 0.05 μm. Such a surface roughness of the outermost layer may however also be conceivable in case of more than one layer.

The anchoring system has also the capacity to form apatite in-situ. By capacity to form apatite in-situ it is hereby meant that the system comprises the components that are necessary for the formation of different types of apatite, hydroxyapatite or fluoride-apatite ((Ca₅(PO₄)₃OH and Ca₅(PO₄)₃F, respectively) for example, and optionally some other biologically favourable phase, and that the system allows for such phases to be formed during and/or after the hydration reaction. The body liquid, which contains hydrogen and dihydrogen-phosphates and hydrogen carbonate ions, interacts with the non-hydrated or partially hydrated material of the coating in formation of the biominerals apatite and in some cases carbonate. Hereby, the advantage is at least attained that apatite need not be added as a separate additive. The ceramic material of the coatings of the implants may further contain water-soluble phosphate or a phase (such as a phosphate salt) that has the capacity to form water-soluble phosphate. The material formed can be said to constitute a chemically bonded ceramic composite that exhibits many advantages as a coating layer on an implant material. The formation of apatite in the material is a sign of the material being bioactive and co-operating with the body. Furthermore, the distribution of apatite will be homogeneous in the material, also in contact zones against biological material. The formation of apatite in such contact zones is especially favourable for the anchoring process. Another advantage for the formation of apatite is that the environment is basic. Since apatite is an endogenous substance, the anchoring system will result in excellent anchoring properties with a very tight union between the implant material and the biological tissue.

Surprisingly, it has been found that a calcium-based cement system comprising water-soluble phosphate or a phase (such as a phosphate salt) that has the capacity to form water soluble phosphate, at a boundary or a gap between a biological tissue and an implant material, not only provides for the formation of a chemically bonded ceramic composite comprising apatite, but also leads to a faster healing of the bone. It has been found that a chemical and biological integration takes place, that leads to an additional surface growth that chemically diminishes the gap between the biological tissue and the implant material, but that also, due to the presence of apatite, will result in a faster biological sealing of the gap. The healing or growing process of the bone is favoured by an early fixation (less micromotion leading to less fibrous tissue) and by the supply of calcium and phosphate and carbonate from the cement-body liquid system. The dissolution-precipitation of the Ca-based system process is able to close large gaps (millimeter size), and by the increase in volume (or mass) related to the formation of hydrates, the volume increase of the contact points with the biological tissue will provide for further early fixation.

Accordingly, calcium is taken from the calcium-based cement system, e.g. a calcium aluminate cement. Below a surface layer of a formed apatite, the content of Ca will therefore be somewhat reduced, which leads to an increased formation of gibbsite phase in the produced ceramic material. The extent of this gibbsite phase may be controlled by the content of Ca and the addition of phosphate in the contact zone.

Another aspect of the formation of hydroxyapatite (formation of HAP), in connection with the general mechanism at hardening comprising dissolving and depositing, is that the system may act to favour healing of damaged bone tissue. Hereby, the biological material that has lost its hard material (its biologically formed apatite) is remineralised by Ca-aluminate reacting with body liquid to form hydrates including apatite. The material is dissolved, i.e. becomes a solution and ions such as calcium, aluminate, phosphate, hydroxyl and optional additives, such as fluoride, are deposited as hydrates in all voids, including those originating from previous bone decay. Also other bone materials can be favoured in healing in a corresponding manner, e.g. related to osteoporosis etc.

Said implant may be any medical, orthopaedic or dental implant. As examples of possible implants, one can mention artificial orthopaedic devices, spinal implants, joint implants, attachment element, bone nails, bone screws, and bone reinforcement plates.

The above-mentioned implants may be manufactured from a ceramic, metallic or polymeric material, preferably a material chosen from the group that consists of titanium, stainless steels, alumina, zirconia and medical grade plastics.

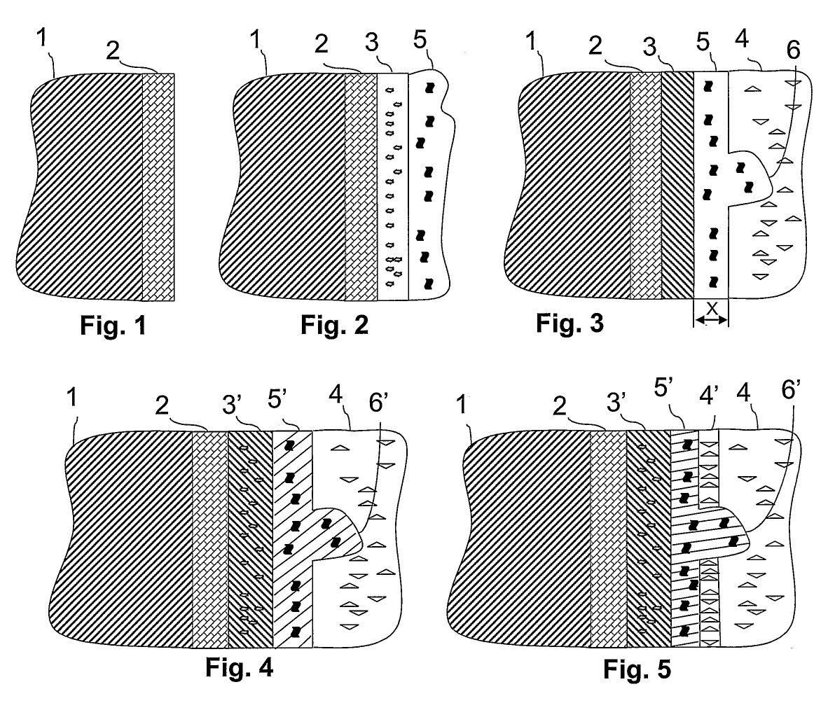

In the drawings, reference number 1 denotes a metal, ceramic or polymeric implant. FIG. 1 shows how a coating layer 2 of a CBC-material has been applied and optionally hydrated.

FIG. 2 shows how an extra, outermost layer 3 has been applied on the coating 2. The coating layer 2 suitably exhibits a thickness of less than 2 μm. The outer layer 3 is thicker (although not apparent from the Figures), but suitably not thicker than 20 μm. The outer layer 3 is composed of non-hydrated CA (without any hydration liquid) that preferably comprises phosphate. FIG. 2 also shows that a paste 5 of CBC-material has been applied onto the outer layer 3, just prior to the implantation operation to take place.

FIG. 3 shows how the implant 1 with the coating layer 2, the outer layer 3 and the paste 5 has been implanted against a biological wall in existing hard tissue, usually bone tissue 4, of the patient. Immediately after the implantation, there is a gap x between the outer surface of the outer layer 3 of the implant and the hard tissue that in average is about 10 μm, which gap always will arise even if the implant is put completely in abutment with the hard tissue. Only point contacts exist. In point contacts (not shown in the figure), however, the outermost layer will during hydration—due to volume/mass increase—enlarge the contact surface. Also, there may be vacuoles 6 in the hard tissue, where the hard tissue is damaged and may have lost its possibility to remineralise. In FIG. 3, it is shown how the implantation system according to present invention, which includes the paste 5, advantageously fills both the gap x and any vacuoles 6.

FIG. 4 shows how the outer, non-reacted layer 3 has hydrated to a hydrated layer 3′, in which case a surface growth of 1-3 μm has normally occurred due to chemical mass growth on the outer layer 3, 3′. This mass growth depends on an uptake of water, body fluid or hydration liquid, in the non-hydrated layer 3. Also the paste 5 has hydrated such that it forms a hydrated layer 5′, also including a part 6′ filling the former vacuole 6.

FIG. 5 shows how the coated implant 1 has been integrated with the hard tissue 4, after healing 4′. The healing and integration will be even faster, if Ca-ions and optionally phosphate/apatite are supplied to the area between the coated implant or implant system and the biological tissue via the coating 2, the outer layer 3 and or the paste 5. The biologically induced growth of new bone tissue 4′ is united with the outer grown layer 3′ and the hydrated paste 5′. In the case the paste and/or the coating are based on slowly resorbable systems, e.g. Ca-silicates, an early fixation is achieved, but in a later stage the hydrated material will be resorbed and exchanged by newly formed tissue. The biologically related growth is positively affected by the presence of hydroxyapatite. The size of the gap x has, according to the above, been diminished by the chemical growth of layers 3′ and 5′, which per se will accelerate the biological filling of new bone tissue 4′. In this case, there had been no growth of new bone tissue in the vacuole 6, since the old bone tissue was damaged and lacked the possibility to remineralise, but it should be understood that vacuoles in other cases still may have the possibility to develop new bone tissue 4′.

### EXAMPLE 1

Titanium dental screw implants with a diameter of 3.70 mm and having a thread length of 5 mm were implanted in the tibia condyl of adult rabbits. These screws, mildly sand-blasted, were used as reference screws (Series D below). Holes were drilled following a dental implantation procedure involving two drilling steps using tools with a greater diameter than that of the implant, followed by creation of threaded holes into which all implants were screwed to the same depth.

Other implant screws, of the same type as the references screws (mildly sand-blasted), were plasma-sprayed, with a calcium aluminate, CaO.Al₂O₃, (Series A) and calcium silicate, CaO.SiO₂ (Series B). Both series were sprayed such that they generated a surface coating having a thickness of about 30 microns on the threaded section. A third series C, were RF-sputtered with a thin CA-coating (approximately 0.2 μm and covered with a thin water-based calcium aluminate paste (having the same composition as used in Series A), which was applied directly before implantation. The CA-paste was an aqueous solution comprising 6.5 g LiCl per litre in order to accelerate the curing of the calcium aluminate. The implants were removed 24 hrs after implantation, the maximum removal torque in Ncm was recorded.

The results show that the implants coated with calcium aluminate provides a faster anchoring to bone than the sand-blasted titanium screws without said coating, see Table 1. The 24 h-values are approximately 100% higher than those of the reference samples.

TABLE 1
Removal torque values after various time periods in
rabbit model. (Standard deviation within brackets)

| | | | Number |
|---|---|---|---|
| | Coating technique | 24 h [Ncm] | of screws |
| A | Plasma-sprayed CA-coating, 30 μm | 7.6 (1.8) | 8 |
| B | Plasma-sprayed CS-coating, 30 μm | 7.2 (1.5) | 8 |
| C | Screws sputtered and dipped in | 7.8 (1.9) | 8 |
| | CA-biocement | | |
| D | Non-coated, sand-blasted titanium screws | 3.8 (1.0) | 8 |

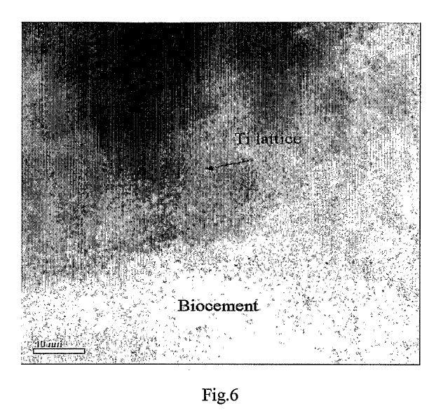

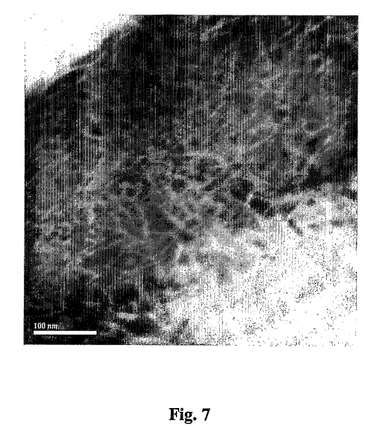

High-resolution TEM of the coating produced with series C, revealed that the contact with the titanium surface was very close, and using a magnification of 600.000 X, to be on the atomic and nano scale (see FIG. 6.).

### EXAMPLE 2

A transmission electron microscopy (TEM) study of the hydrate grain size was performed on plasma-sprayed coatings of hydrated CaOAl₂O₃.

Metallic implants were put in the femur of rabbits for 24 h. The rabbits were then terminated and the implant fixated and embedded. To obtain TEM samples of the hydrated coatings, focused ion beam microscopy (FIB) was used. Cross-sections of the metal-coating interface were produced via cutting with a diamond saw and polished to 0,25 micron using a cloth and diamond paste. TEM-samples of five by five micron were produced from the cross-sections using the FIB. The samples were then imaged in annular dark field STEM mode in a 200 keV FEG TEM (Jeol).

The hydrates were plate- or needle-shaped and had a grain size of below 100 nm, see FIG. 7.

### EXAMPLE 3

A chemically active surface was produced on an inert alumina implant by pressing a layer of CaOAl₂O₃ onto the alumina surface, followed by a heat treatment at 1100° C. for 6 h. Examination of the surface composition after heat treatment with X-ray diffraction, showed that only crystalline CaOAl₂O₃ was present on the surface. The adhesion between the CaOAl₂O₃ layer and the implant was very strong as tested with scratch testing, and no delamination of the coating occurred.

1. A coated implant for in vivo-anchoring to a biological tissue or another implant, which coated implant comprises an implant having a pre-treated surface and on said pre-treated surface one or more layers of mainly non-hydrated chemically bonded ceramic material, characterised in that each layer of said ceramic material independently comprises a first binder phase selected from the group consisting of aluminates, silicates, phosphates, sulphates and combinations thereof, and that said ceramic material is chemically and/or mechanically bound to said implant.

2. A coated implant according to claim 1, characterised in that the first binder phase comprises cations selected from the group consisting of Ca, Sr and Ba.

3. A coated implant according to claim 2, characterised in that the cations are Ca-cations.

4. A coated implant according to claim 3, characterized in that the first binder phase comprises calcium aluminates.

5. A coated implant according to claim 4, characterized in that the first binder phase comprises one or more of the phases 3CaO.Al₂O₃, 12CaO.7Al₂O₃ CaO.Al₂O₃, CaO.Al₂O₃ and CaO.6Al₂O_{3.}

6. A coated implant according to claim 1, characterised in that the ceramic material further comprises water-soluble phosphate or a phase (such as a phophate salt) that has the capacity to form water-soluble phosphate.

7. A coated implant according to claim 1, characterised in that said one or more non-hydrated layers have a porosity below 50%.

8. A coated implant according to claim 1, characterised in that the surface roughness of the pre-treated surface of the implant has a Ra-value of less than 10 μm, but not smaller than 0.5 μm.

9. A coated implant according to claim 1, characterised in that the number of layers of the coating is 1-5.

10. A coated implant according to claim 1, characterised in that an innermost layer has a thickness in the interval from nanometer level to less than 10 μm.

11. A coated implant according to claim 1, characterised in that an outermost layer has a surface treated to a surface roughness of Ra<20 μm, but not smaller than 0.5 μm.

12. A coated implant according to claim 1, characterised in that it comprises at least two layers and that each layer outside the innermost one independently has a thickness of less than 50 μm, but not smaller than 5 μm.

13. A coated implant according to claim 1, characterised in that said implant is a medical, orthopaedic or dental implant, such as an artificial orthopaedic device, a spinal implant, a joint implant, an attachment element, a bone nail, a bone screw, and a bone reinforcement plate.

14. A coated implant according to claim 1, characterised in that said implant is of a ceramic, metallic or polymeric material.

15. A coated implant according to claim 14, characterised in that said implant material has been selected from titanium, stainless steels, alumina, zirconia and medical grade plastics.

16. A coated implant according to claim 1, characterised in that the implant surface is oxidized.

17. A coated implant according to claim 16, characterised in that said oxide is a double oxide of titanate, silicate or aluminate type.

18. A coated implant according to claim 1, characterised in that said mechanical binding to the implant is achieved by sub-micron size crystallites of hydrates precipitated on the surface of said implant.

19. A coated implant according to claim 18, characterised in that the crystallite size is less than 100 μm.

20. A coated implant according to claim 1, characterised in that the powdered mainly non-hydrated ceramic material has a particle size of 0.1 to 20 μm.

21. A method of manufacturing a coated implant according to claim 1, which method comprises the steps of:
pre-treating the surface of an implant,
applying on said pre-treated surface one or more layers of mainly powdered non-hydrated ceramic material, which layers independently comprises a first binder phase selected from the group consisting of aluminates, silicates, phosphates, sulphates and combinations thereof, and
optionally pre-hydrating said ceramic material by contacting it with a curing liquid or body fluid,
thereby forming a chemical and/or mechanical bond between the ceramic material and said implant.

22. A method according to claim 21, characterised in that said pre-treatment is selected from a group consisting of oxidation including low-temperature oxidation, thermal treatment including solid state diffusion and ion bombarding, etching including the use of salt melts, calcination, sand-blasting and grinding.

23. A method according to claim 21, characterised in that the surface roughness of the implant after pre-treatment has a Ra-value of less than 10 μm, but not smaller than 0.5 μm.

24. A method according to claim 23, characterised in that the innermost layer of the coating is applied on the implant surface by any of the following techniques: thermal spraying, flame spraying, Electro Deposition Spraying (EDS), plasma spraying, dipping and spin coating.

25. A method according to claim 23, characterised in that when the surface roughness of the implant has a Ra-value of less than 1 μm, but not smaller than 0.05 μm, the innermost layer of the coating is applied on the implant surface by any of the following techniques: Chemical Vapor Deposition (CVD), Physical Vapor Deposition (PVD), laser techniques including laser cladding, Electrolytic Deposition (ED), and sol-gel techniques.

26. A method according to any of claims 25, characterised in that when the coating only comprises one layer, said layer is applied using Physical Vapor Deposition (PVD).

27. A method according to claim 21, characterised in that said one or more layers of the coating are thinned, preferably by a process selected from the group consisting of grinding, sand blasting, dry etching and chemical treatment including dissolution.

28. A method according to claim 27, characterised in that in connection with said thinning, a partial densification of said one or more layers is performed, preferably by drying up of particles and precipitation including sol-gel techniques.

29. A method according to claim 21, characterised in that the pre-hydration is performed by dipping, spraying, spin coating or tape casting the coated implant in/with such an additional hydration liquid.

30. A method according to claim 21, characterised in that the powdered, mainly non-hydrated ceramic material, has a particle size of 0.1 to 20 μm.

31. A ceramic paste, characterised in that it comprises a powdered calcium-based binder of aluminate and/or silicate and a hydration liquid.

32. A ceramic paste according to claim 31, characterised in that it has the form of granules of a size below 1 mm and a granule compaction density above 35%.

33. A ceramic paste according to claim 32, characterised in that the granules have a mean size of at least 30 μm, but 250 μm at the most.

34. A ceramic paste according to claim 31, characterised in that it comprises an organic additive, preferably a hydrophilic polyacrylic and/or polycarboxylate compound.

35. An implantation kit for in vivo-anchoring an implant to a biological tissue or another implant, comprising the coated implant according to claim 1 and optionally a curing liquid capable of hydrating the binder phase of the coated implant and a paste according to claim 31, wherein the ceramic powder and hydration liquid of the paste are kept separately.
